(19) United States
(12) Patent Application Publication
(10) Pub. No.: US 2009/0131757 A1
(43) Pub. Date: May 21, 2009

(54) MULTI MODE PATIENT MONITOR

(75) Inventor: Raghavan Jayaram, Bangalore (IN)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/944,044

(22) Filed: Nov. 21, 2007

(51) Int. Cl. A61B 5/00

(52) U.S. Cl. 600/300

(57) ABSTRACT

A multimode patient monitor is disclosed herewith. The patient monitor comprises: a display, wherein the patient monitor operates in a normal mode wherein monitored patient status is displayed using normal display characteristics of the patient monitor and also operates in a night mode wherein monitored patient-status is displayed using modified display characteristics, the modified display characteristics of the patient monitor being modified based on at least available light level in a patient room. The patient monitor is further provided with an interface that allows configuration of the patient monitor either in the normal mode or in the night mode.

### FIELD OF THE INVENTION

The present invention relates generally to display systems. More particularly, this invention relates to, patient monitor configured to have multiple modes of indication.

### BACKGROUND OF THE INVENTION

When providing medical care to patients, it is frequently necessary to monitor the patient using medical diagnostic instruments. One type of instrument, the patient monitor, is capable of monitoring the patient to acquire electrocardiogram data, cardiac output data, respiration data, pulse oximetry data, blood pressure data, temperature data and other parameter data. Various patient monitors are configured to indicate the patient status visually or audibly to a caretaker. Different forms of indication could be used to indicate the patient status based on the clinical nature of the patient, criticality of patient, etc. Once the patient monitor is configured to indicate the patient status in a particular form or mode, the patient monitor keeps on indicating the patient status in the selected mode of the patient monitor. For example, a patient monitor may be configured to generate a loud alarm upon noticing a change in blood pressure data. This variation in blood pressure data may not be critical, but since the patient monitor is configured to generate an alarm, it generates an alarm, irrespective any condition such as urgency, time of the day, or presence of other patients in the same room. This kind of indication could cause some inconvenience. The patient monitors are generally placed on bedside and generates alarm in visual or audible form irrespective of the time of the day or nature of the alarm and this may cause inconvenience to the patients. If there are many hospital beds in a single room, the other patients will be disturbed by an alarm generated based on the patient status of a nearby patient. This is more severe in the night as the patient monitors are generally configured to use high intensity displays and loud alarms to convey the patient status to the caretaker.

Thus, there exists a need to provide a method and system for displaying the patient status in a patient monitor without causing inconvenience to the patient or the caretaker, at the same time without affecting the patient care.

### SUMMARY OF THE INVENTION

The above-mentioned shortcomings, disadvantages and problems are addressed herein which will be understood by reading and understanding the following specification.

One embodiment of the present invention provides a multi-mode patient monitor. The patient monitor comprises: a display, wherein the patient monitor operates in a normal mode wherein monitored patient status is displayed using normal display characteristics of the patient monitor, and also operates in a night mode wherein monitored patient status is displayed using modified display characteristics, the modified display characteristics of the patient monitor being modified based on at least available light level in a patient room; and an interface that allows configuration of the patient monitor either in the normal mode or in the night mode.

In another embodiment, a patient monitor is disclosed. The patient monitor comprises: a detector for detecting patient status; an indicator including visual indicator and audio indicator configured to indicate the patient status; a light detector configured to detect a light level corresponding to available light in a patient room; and a mode selector configured to select a mode of indication of the patient monitor with respect to the light level.

In yet another embodiment, a method of operating a patient monitor in night mode is provided. The method comprises: switching the patient monitor to a night mode based on at least availability of light level in a patient room; and modifying at least one characteristic of a patient monitor display upon switching to the night mode, the characteristic including at least one of volume level and a display parameter.

In yet another embodiment of the invention, a method of indicating patient status in a patient monitor is disclosed. The method comprises: identifying a mode of indication based on at least light availability in a patient room; and indicating the patient status using the identified mode of display.

In yet another embodiment of the invention, a method of patient monitoring is disclosed. The method comprises: providing a multi-mode patient monitor for monitoring status of a patient; checking at least light ambiance in a patient room; selecting a mode of display based on the light ambiance; and indicating the patient status using the selected mode.

Various other features, objects, and advantages of the invention will be made apparent to those skilled in the art from the accompanying drawings and detailed description thereof.

### BRIEF DESCRIPTION OF THE DRAWINGS

A clear conception of the advantages and features constituting inventive arrangements, and of various construction and operational aspects of typical mechanisms provided by such arrangements, are readily apparent by referring to the following illustrative, exemplary, representative, and non-limiting figures, which form an integral part of this specification, in which like numerals generally designate the same elements in the several views, and in which:

FIG. 1 is a generic block diagram of a multi mode patient monitor as described in an embodiment of the invention;

FIG. 2 is a detailed block diagram of a multi mode patient monitor as described in an embodiment of the invention;

FIG. 3 is a flowchart illustrating a method of operating a patient monitor in night mode as described in an embodiment of the invention;

FIG. 4 is a flowchart illustrating actions involved in switching mode of indication of a patient monitor to night mode as described in an embodiment of the invention;

FIG. 5 is a flowchart illustrating actions involved in switching mode of indication of a patient monitor to normal mode from night mode of indication as described in an embodiment of the invention;

FIG. 6 is a flowchart illustrating a method of indicating patient status in a patient monitor as described in an embodiment of the invention; and

FIG. 7 is a flowchart illustrating a method of patient monitoring using a patient monitor as described in an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

In various embodiments, a method and system for displaying patient status in a patient monitor based on the light availability in the room is disclosed. The patient monitor is provided with different sets of volume levels and/or display characteristics corresponding to different modes and is configured to select a desired set of volume levels and/or display characteristics upon identifying the light level.

In an embodiment, the invention provides a patient monitor configured to identify a light level in the patient room and select display characteristics corresponding to the detected light level and display patient status using the selected display characteristics.

In an embodiment a patient monitor with a night mode of display is provided, wherein the monitor is configured to display patient status with low intensity, subdued color, low brightness and/or low volume.

In an embodiment a patient monitor is provided with multi mode display options, wherein a user may manually select the desired mode on display based on clinical nature of patient, time of the day, nature of patient such as neonates or light phobic patients, etc. or based on presence of other patients in the same room. In an embodiment, in anticipation of a situation the caretaker may set the mode of display. For example, if the patient is given any sedation, the caretaker may select the mode of display as night mode of display. For a patient who is sensitive to monitor display, the blinking display of alarms/parameters on a monitor might trigger seizures on patients with photosensitive epilepsy.

In an embodiment the patient monitor may automatically change the mode of operation based on the light availability in the patient room. In alternative embodiments the patient monitor may be configured to select the mode of display automatically, based on the type of patient, criticality of patient, time of the day or based on presence of other patients in the same room.

In another embodiment the patient monitor is configured to be provided with different sets of display characteristics and, based on the light availability, may select a desired set of display characteristics. Alternately, an interface may select the display characteristics. In another embodiment the monitor may be configured to define display parameters in real time based on light availability, rather than selecting from the available display characteristics of display. Also the user may configure the patient monitor by setting display characteristics based on the light availability or in anticipation of a situation.

In an embodiment the invention provides a mechanism to check different qualifying parameters before changing the mode of operation, so that unnecessary switchover between different modes can be avoided.

In an embodiment, a monitor in alarm condition can be distinguished easily, since its display characteristics in normal or subdued display mode will be markedly different from all other monitors in the room, which are in night mode. This helps the caregiver to easily locate the patient in alarm condition.

In an embodiment a preset time level may set indicating the night mode and normal mode and the monitor may select the mode in accordance with the preset time. Alternately, the patient monitor could learn a circadian rhythm, i.e. register the maximum and minimum light levels from past few days and adapt the threshold accordingly.

While the present technique is described herein with reference to patient monitors, it should be noted that the invention is not limited to this or any particular application or environment. Rather, the technique may be employed in a range of medical instruments, especially medical diagnostic instruments having a monitor for display.

The term patient status referred in the description includes patient status along with monitored parameters, patient identification information, etc.

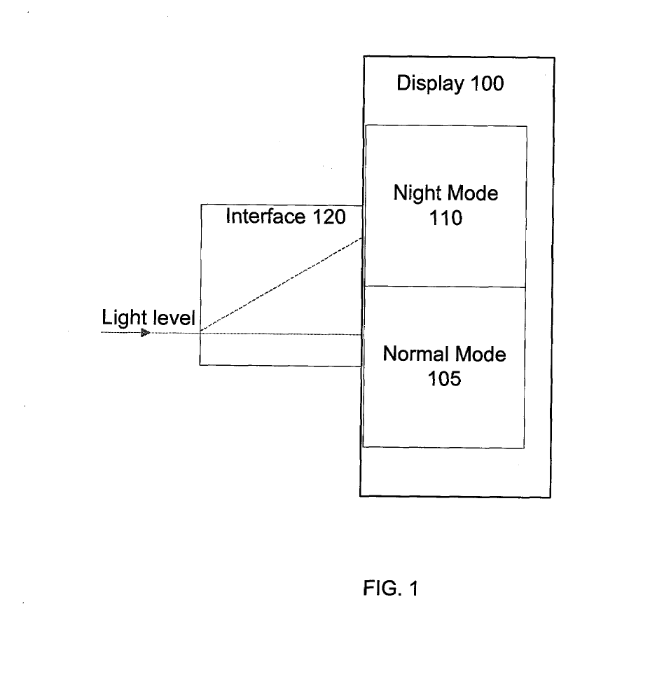

FIG. 1 is a generic block diagram of a multi mode patient monitor as described in an embodiment of the invention. The multi mode patient monitor is provided with a patient monitor display, in the form of an audio and/or visual indicator. The multimode patient monitor is configured to indicate patient status in a display 100 having a normal mode 105 or in a night mode 110 of operation. The multi mode patient monitor, herein after referred as monitor or patient monitor, is configured to monitor various patient statuses corresponding to different clinical factors and display the monitored status along with monitored parameters and/or patient related information. The clinical factors may include cardiac output data, respiration data, pulse oximetry data, blood pressure data, temperature data and other parameter data, and examples of patient status may include “Normal”, “Alert”, “Caution”, “Serious”, “Severe”, “Danger” or “Critical”, etc. The monitor provided with the display 100 is configured to indicate the detected patient status in different modes based on at least availability of light in a patient room. In an example, the display 100 is configured to operate in normal mode 105 or in night mode 110 based on the light availability in the patient room. An interface 120 is configured to select the mode of indication of the patient monitor. In an embodiment, a user or caretaker may decide the mode of operation of the patient monitor and convey the selected mode through the interface 120. The user may configure the display 100 to indicate the patient status in the selected mode. In an embodiment the interface 120 may automatically select the mode of indication based on the light availability and may configure the monitor to indicate the patient status based on the selected mode of operation. In an embodiment the interface 120 may be operably coupled to the monitor. In alternative embodiments the interface 120 may be provided as a part of the monitor. Once the mode of indication is selected, the monitor will select the display characteristics and will indicate the patient status accordingly. The display 100 may be provided with different set of display characteristics and the monitor or interface 120 may select the display characteristics corresponding to the selected mode. Alternately the patient monitor may define the display characteristics in real time based on the mode selected. If the mode of indication is normal mode 105, the patient monitor is configured to indicate the patient status using the normal display characteristics of the display 100. Alternatively, if the patient monitor is selected to be operated in night mode, the patient monitor is configured to alter the display characteristics such as the intensity, brightness or color of patient monitor display and reduce the volume level of audio indicator. The display 100 may select different display characteristics available in the monitor corresponding to different modes or the patient monitor may be configured to modify the display characteristics automatically in real time based on the mode selection. In an embodiment the night mode is configured to include different modes such as subdued mode, wherein the patient status is conveyed using subdued display characteristics.

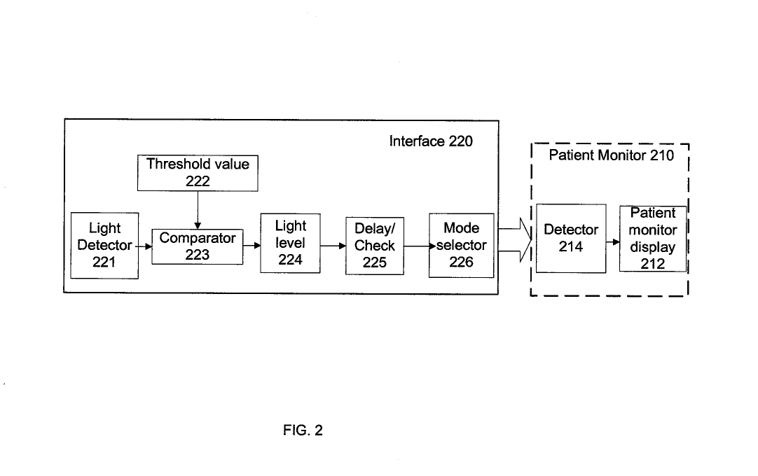

FIG. 2 is a detailed block diagram of a multi mode patient monitor as described in an embodiment of the invention. The patient monitor 210 is a multimode patient monitor configured to indicate the patient status in different modes based on light availability in a patient room. The patient monitor 210 is provided with a detector 214 for detecting the patient status and with a patient monitor display 212 having a visual indicator and an audio indicator. In an embodiment the patient monitor 210 is configured to be operable through an interface 220. The interface 220 could be provided as a part of the monitor 210 or can be operably connected to the monitor 210.

In an embodiment the interface 220 is configured to decide the mode of indication of the patient monitor display 212 based on the light availability in patient room. A light detector 221 is provided for detecting the light available in the patient room. The light detector 221 can be any light detecting circuit including an array of sensors. For each patient monitor depending on different parameters such as clinical situation, clinical parameters, climatic condition, nature of expected patient status, nature of patients, etc, may set a threshold value 222 of light level, for changing the mode of indication. The threshold value 222 and the available light detected by the light detector 221 are fed to a comparator 223. A light level 224 is determined based on the comparison result. If the available light in the patient room is less than the threshold value 222, the light level 224 is assigned as low light level. If the available light in the patient room is more than the threshold value 222, the light level may be designated as high light level. Once the light level 224 is identified, the interface 220 will wait for a preset time to check whether there is any change in the light level 224 of the patient room. A delay circuit 225 introduces this delay. For example, upon detecting a change in the light level 224, the interface 220 will wait for a preset time before changing the mode of indication of the patient monitor 210 and this will avoid unnecessary switching of the patient monitor 210 between different modes. Further during the wait period, different checks can be done to improve the performance quality of the patient monitor 210. In an example, during this period the patient monitor 210 directly or through the interface 220 may check for conditions such as generation of any alarm signal or any user activity at the patient monitor 210. If any of these events occur during the preset time, the patient monitor 210 will not change its mode of indication. In an embodiment the user may provide the mode of operation and/or set display characteristics of the patient monitor through a touch panel or any other user interface. Once the light level is changed and remains at the new level for the preset time, a mode selector 226 will select the mode of indication of the patient monitor 210. Once the light level 224 is changed and the preset time is elapsed, the mode selector 226 provided may convey the mode of indication to the patient monitor 210. The patient monitor 210 is configured to select or alter the display characteristics of the patient monitor display 212 upon receiving the mode selection from the interface 220. The patient monitor display 212 is configured to include audio and/or visual indicator and altering the display characteristics may include changing the intensity, color or brightness level of the visual indicator and/or reducing the volume level of the audio indicator.

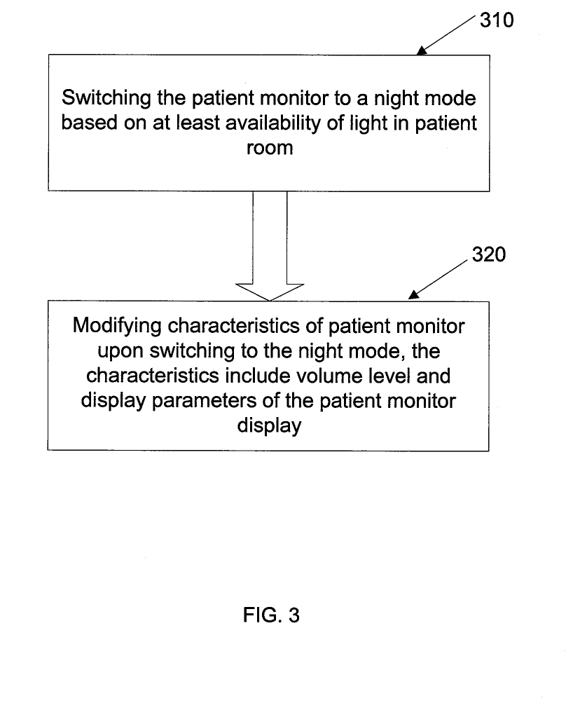

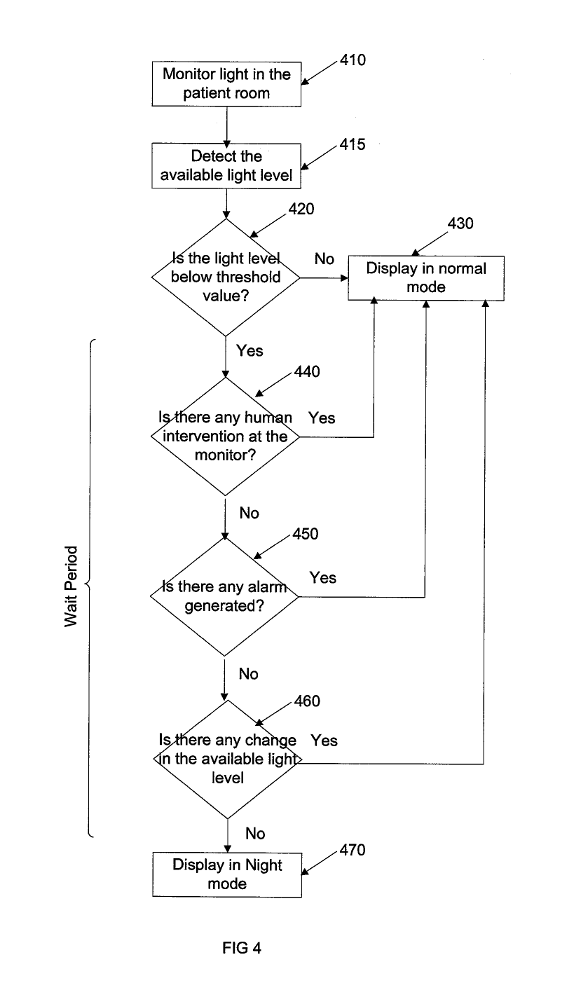

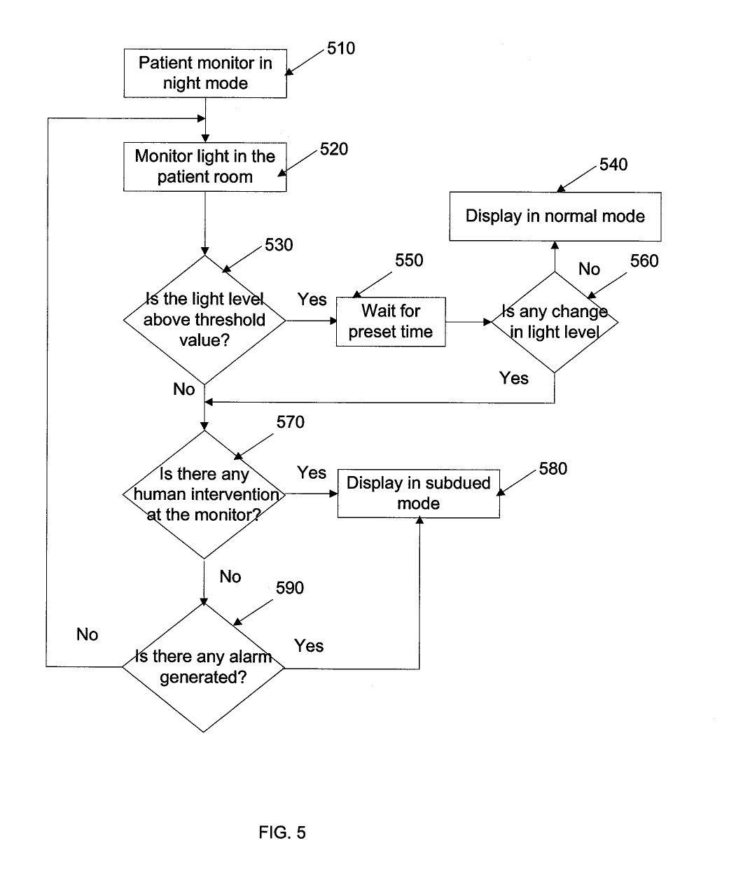

FIG. 3 is a flowchart illustrating a method of operating a patient monitor in night mode as described in an embodiment of the invention. At step 310, the patient monitor is switched to night mode operation, based on the light availability in the patient room. The step includes monitoring available light in the patient room and generating a light level with reference to a threshold value. If the light level in the patient room is below the threshold value, then the patient monitor is configured to switch to night mode of indication. The patient monitor is configured to wait for a preset time before switching into the night mode, even after detecting the light level in the patient room as below the threshold value. During the preset time the patient monitor may check a set of conditions to improve the efficiency of the patient monitor operation. The sequence of events needs to be considered while switching to night mode of indication is explained with reference to FIG. 4. At step 320, the display characteristics of patient monitor are modified. The characteristics of the patient monitor that may be altered is, display parameters of visual indicator and/or volume level of the audio indicator. The display parameters of visual indicator may include brightness, intensity or color of the patient monitor display. In the night mode the patient status is displayed using low intensity, low brightness and subdued color display or using low volume. In an example, the patient monitor is provided with different sets of display characteristics and based on the selected mode the patient monitor may select the desired display characteristics. Alternately the monitor may define desired set of display characteristics in real time corresponding to the selected mode. The user may also suggest display characteristics based on the mode or in anticipation of a mode selection. For example, if a patient is going to bed, the caretaker may select the night mode of operation. Further if the caretaker is anticipating an alarm, the caretaker may set the display characteristics accordingly. The night mode feature is optional and can be disabled if not required. Upon detecting the light level as above the threshold value the patient monitor is configured to switch back to the normal mode of indication and various sequences involved in this process is explained with reference to FIG. 5.

FIG. 4 is a flowchart illustrating actions involved in switching mode of indication of a patient monitor to night mode as described in an embodiment of the invention. At step 410, light available or ambient light in the patient room is monitored. An ambient light sensor may be provided. In an embodiment, the monitor may receive ambient light intensity information from a remote sensor. In an embodiment the ambient light may be measured using more than one sensor and utilize the information in decision making. For example, one sensor may be accidentally pointed towards a point such as light source or some other object may shade one sensor. At step 415, based on the light availability, a suitable light level is identified and this could be achieved by comparing the light available in the patient room with a threshold value. At step 420, a check is made to identify whether the light level is above or below the threshold level. If the light level is above the threshold level, the patient status is displayed in normal mode using the normal display characteristics of the patient monitor as shown at step 430. If the light level is below the threshold level, the patient monitor is configured to wait for a preset time before switching the mode of indication. During this preset time, the patient monitor is configured to perform operations indicated at steps 440 to 460. At step 440, a check is made to find out whether there is any human intervention at the monitor. If there is a human intervention or any user activity at the monitor, the monitor is configured to display the patient status in normal mode as at step 430. If there is no human activity detected at the patient monitor, a check 450 is made to identify whether there is any alarm generated based on patient status. If there is an alarm generated, the patient status is displayed using normal mode of indication as at step 430. The steps 440 and 450 can be performed simultaneously or in any order. Further one could include additional checks such as if the patient status is detected as critical, the mode of indication should be normal mode irrespective of any other condition. In the absence of any alarm, the patient monitor checks, at step 460, whether there is any change in the light level and if there is a change, the patient monitor is configured to display the patient status in normal mode as at step 430 and if there is no change in the light level, the patient status is displayed in night mode as at step 470. In short, after detecting light level as below the threshold value, the patient monitor will wait for a preset time before changing the mode of operation. This ensures that momentary changes in light level do not trigger mode switching on the monitor. During the preset time the patient monitor will check for any human intervention or alarms at the monitor, or for any change in the light level and if not the patient monitor is configured to change its mode of indication.

FIG. 5 is a flowchart illustrating actions involved in switching mode of indication of a patient monitor to normal mode from night mode of indication as described in an embodiment of the invention. At step 510, the patient monitor is configured to indicate the patient status in night mode of indication. The patient monitor will detect available light in patient room continuously as at step 520. This step includes identifying a light level by comparing the available light in the patient room with a threshold value. Once the monitor identifies a change in the light level in the room at step 530, a check is made to determine whether the light level is above the threshold value or not. If the light level is above the threshold value, the patient monitor waits for a preset time as at step 550. Once the preset time elapsed, the patient monitor checks for any change in the light level at step 560 and if there is no change in the light level the patient monitor displays the patient status in normal mode as at step 540. If there is a change in the light level, indicating that the light level is below the threshold value, the monitor will follow the steps 570-590, as that is followed in the event of light level below the threshold value.

At step 530, if the light level is detected as below the threshold value, or the patient monitor is still in night mode, at step 570, the patient monitor will check for any human intervention at the monitor. If there is a human intervention or any user activity detected at the monitor during night mode operation of the monitor, the monitor is configured to switch to a subdued mode as at step 580. If there is no human intervention detected, the patient monitor will check for any alarm generated based on the patient status as at step 590. If there is an alarm generated the patient monitor will switch to a subdued mode, wherein the intensity and sound level of the display can be in a subdued form decided by the user or the monitor. The steps 570 and 590 could be performed simultaneously or in any sequence. In subdued mode the monitor or user may set the display parameters of the patient monitor display. The user may do so based on the clinical situation, patient's nature or similar other conditions. Else the patient monitor may be provided with a set of display characteristics corresponding to the subdued mode and the patient monitor may select the same upon identification of subdued mode. If there is no alarm generated then the patient monitor is configured to continue to operate in night mode and continue to monitor the light in the room as at step 520.

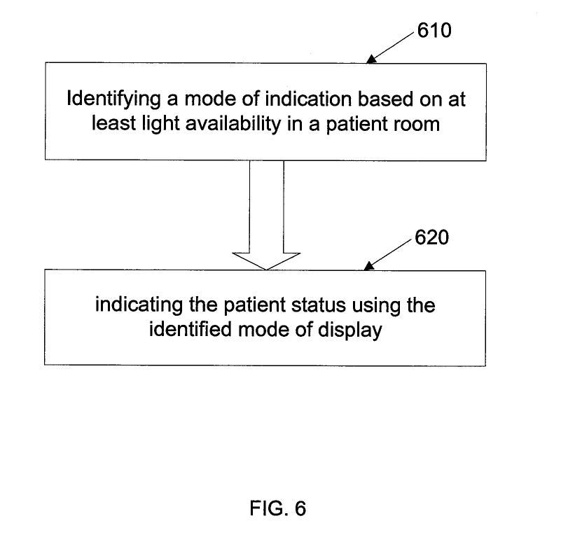

FIG. 6 is a flowchart illustrating a method of indicating patient status in a patient monitor as described in an embodiment of the invention. At step 610, a mode of indication of the patient monitor is selected based on the light availability in the patient room. The step includes configuring the patient monitor to modify its display characteristics based on the mode selected. In an example the patient monitor may be configured to display the patient status in normal mode or night mode. The normal mode is selected if the patient room has sufficient light and the night mode is selected when the light available in the patient room is lower than a threshold value. The patient monitor may be provided with different sets of display characteristics corresponding to different modes and based on the mode selection corresponding display characteristics may be selected. Alternately the caretaker may set display characteristics based on the mode selection or in anticipation of a mode selection. Further the monitor may derive different display characteristics in real time based on the light level. At step 620, the patient status is displayed using the selected mode. The night mode of indication includes dim light, low intensity and subdued colors and reduced volume level. In the night mode there could be other alternative modes based on some conditions. For example, in the night mode, if the patient monitor is detected any user input at the monitor or near the monitor, the monitor may switch to a subdued mode of display. Similarly if the patient monitor detects a patient status and an alarm need to be raised based on the patient status, the monitor may switch to a subdued mode of indication. In the subdued mode the patient monitor is configured to display the patient status using a set of display characteristics provided by the user or selected from the patient monitor. For example, the caretaker may configure the patient monitor to return to normal mode, if the patient status is “critical” and continue in night mode if the patient status is “normal”.

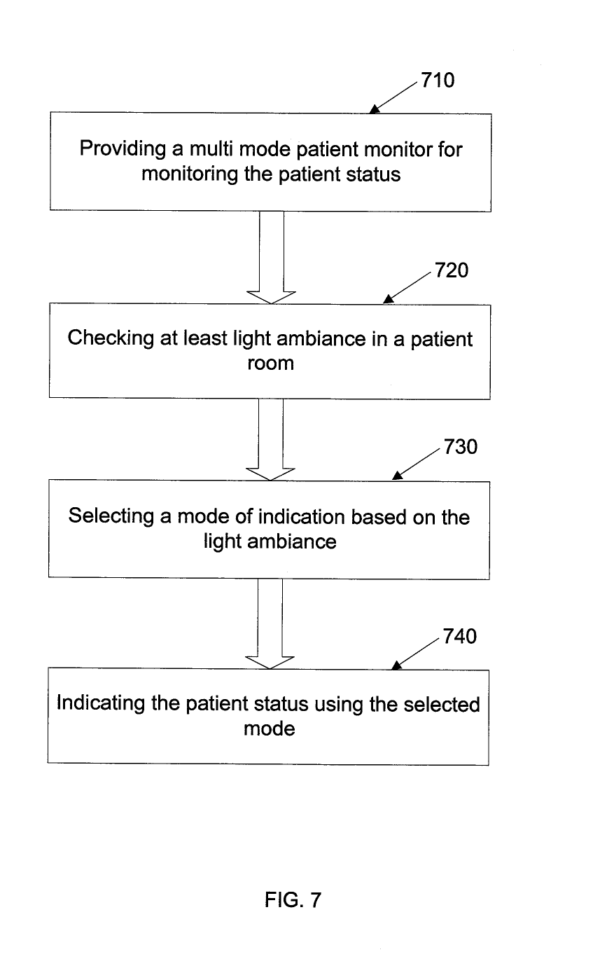

FIG. 7 is a flowchart illustrating a method of patient monitoring by a patient monitor as described in an embodiment of the invention. At step 710, a multi mode patient monitor is provided for monitoring the patient status of patient continuously. The multi mode patient monitor is configured to display the patient status in different modes, based on at least the light availability in patient room. In an example the patient monitor may be provided with different set of parameters corresponding to different light levels and based on the light level selected, desired set of display characteristics may be selected. At step 720, light ambiance in the patient room is checked. This can be achieved by providing light detectors on the patient monitor or some where in the room operably connected to the monitor. At step 730, a mode of indication is selected based on the light level in the patient room. In an example a threshold value may be set to identify the light level in the patient room. At step 740, the patient status is indicated using the selected mode of indication.

Some of the advantages of the invention include reducing the disturbance to the patients caused by the high intensity displays and loud alarms in the patient monitors. The patient monitor displays the patient status without disturbing the patients, but still not compromising on the patient care.

Thus various embodiments of the invention disclose a patient monitor with different modes of indication, the mode of indication being selected based on the light availability in patient room.

As used herein, an element or step recited in the singular and proceeded with the word “a” or “an” should be understood as not excluding plural said elements or steps, unless such exclusion is explicitly recited. Furthermore, references to “one embodiment” of the present invention are not intended to be interpreted as excluding the existence of additional embodiments that also incorporate the recited features.

Exemplary embodiments are described above in detail. The assemblies and methods are not limited to the specific embodiments described herein, but rather, components of each assembly and/or method may be utilized independently and separately from other components described herein. Further the steps involved in the workflow need not follow the sequence in which there are illustrated in figures and all the steps in the work flow need not be performed necessarily to complete the method.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A patient monitor comprising:
a display, wherein the patient monitor is configured to operate in a normal mode wherein monitored patient status is displayed using normal display characteristics of the patient monitor, and is also configured to operate in a night mode wherein monitored patient status is displayed using modified display characteristics, the modified display characteristics of the patient monitor being modified based on at least available light level in a patient room; and
an interface that allows configuration of the patient monitor either in the normal mode or in the night mode.

2. A patient monitor as claimed in claim 1, wherein the patient monitor is configured to identify the mode of operation as the normal mode upon detection of the light level in the patient room above a threshold level and as the night mode upon detection of the light level in the patient room below the threshold level.

3. A patient monitor as claimed in claim 1, wherein the patient monitor is configured to select a set of display characteristics corresponding to the selected mode.

4. A patient monitor as claimed in claim 1, wherein the patient monitor is configured to wait for a preset time after detecting a change in the available light level and before changing the mode of operation.

5. A patient monitor as claimed in claim 4, wherein the patient monitor checks during the preset time for any user activity at the patient monitor or for any alarms generated based on the patient status or for any change in the light level.

6. A patient monitor as claimed in claim 1, wherein operating in the night mode includes modifying the display characteristics of the patient monitor by reducing volume of an audio indicator and reducing at least one of brightness, contrast or color of a visual indicator.

7. A patient monitor as claimed in claim 1, wherein the interface includes a switch operable by a user for changing the mode of operation of the patient monitor.

8. A patient monitor as claimed in claim 1, wherein the interface includes a light detector to detect the light level available in the patient room and to change the mode of indication automatically based on the available light level.

9. A patient monitor as claimed in claim 1, wherein the patient monitor is configured to switch to a subdued mode upon detection of any user activity at the patient monitor or generation of any alarm based on the patient status while the patient monitor is in the night mode.

10. A patient monitor comprising:
a detector for detecting patient status;
an indicator including visual indicator and audio indicator configured to indicate the patient status;
a light detector configured to detect a light level corresponding to available light in a patient room; and
a mode selector configured to select a mode of indication of the patient monitor with respect to the light level.

11. A patient monitor as claimed in claim 10, wherein the light detector is configured to identify a light level in the patient room as above or below a threshold level.

12. A patient monitor as claimed in claim 11, further comprising an interface, wherein the interface is configured to switch the mode of operation to a night mode upon detection of light level in the patient room as below the threshold value, and wherein the night mode operation includes decreasing volume level of the audio indicator and at least one of brightness, contrast or color of the visual indicator.

13. A patient monitor as claimed in claim 11, wherein the patient monitor is configured to wait for a preset time after detecting a change in the light level and before changing the mode of indication.

14. A patient monitor as claimed in claim 13, wherein the patient monitor is further configured to switch to a normal mode upon detecting the light level in the patient room as above the threshold value.

15. A patient monitor as claimed in claim 14, wherein the patient monitor is further configured to switch to a subdued mode from a night mode upon detection of any user activity at the patient monitor or any alarms generated based on the patient status.

16. A method of operating a patient monitor in a night mode comprising:
switching the patient monitor to the night mode based on at least availability of light level in a patient room; and
modifying at least one characteristic of a patient monitor display upon switching to the night mode, the characteristic including at least one of volume level and a display parameter.

17. A method as claimed in claim 16, wherein the step of switching the patient monitor to the night mode comprises: detecting the light level in the room as below a threshold level.

18. A method as claimed in claim 17, wherein the step of switching the patient monitor to the night mode further comprises: waiting for a preset time after determining the light level and before switching to the night mode.

19. A method as claimed in claim 18, further comprising: checking for any user activity at the patient monitor or for any alarms generation based on a patient status, during the preset time or for any change in the light level.

20. A method as claimed in claim 18, wherein the step of modifying characteristics includes: selecting a set of display characteristics corresponding to the selected mode, the selection including at least one of reducing volume level of a patient monitor display, and changing at least one of brightness, contrast or color of the patient monitor display.

21. A method of indicating patient status in a patient monitor, comprising:
identifying a mode of indication based on at least light availability in a patient room; and
indicating the patient status using the identified mode of display.

22. A method as claimed in claim 21, wherein the step of identifying a mode of indication includes identifying the mode of indication as normal mode or night mode, the night mode being configured to use lesser volume and dim light while indicating the patient status.

23. A method as claimed in claim 22, wherein the night mode includes a subdued mode representing a night mode with detected human intervention on monitor activity or an alarm generation based on the patient status.

24. A method of patient monitoring comprising:
providing a multi mode patient monitor for monitoring status of a patient;
checking at least light ambiance in a patient room;
selecting a mode of display based on the light ambiance; and
indicating the patient status using the selected mode.

25. A method as claimed in claim 24, wherein the step of providing the multi mode patient monitor comprises: configuring the patient monitor to operate in a normal mode or in a night mode or in a subdued mode based on the light ambiance, the night mode and subdued mode modifies display parameters of patient monitor display.
